(19) United States
(12) Patent Application Publication
(10) Pub. No.: US 2024/0342455 A1
(43) Pub. Date: Oct. 17, 2024

(54) MICRONEEDLE AND ARRAY AND METHOD OF FABRICATING SAME

(71) Applicant: UTI Limited Partnership, Calgary (CA)

(72) Inventors: Thomas M. LIJNSE, Calgary (CA); Colin DALTON, Calgary (CA); Syed Kazim HAIDER, Calgary (CA)

(21) Appl. No.: 18/701,047

(22) PCT Filed: Oct. 17, 2022
(86) PCT No.: PCT/CA2022/051525

§ 371 Date: Apr. 12, 2024

Related U.S. Application Data
(60) Provisional application No. 63/256,272, filed on Oct. 15, 2021.

(51) Int. Cl. A61M 37/00

(52) U.S. Cl. CPC A61M 37/0015; A61M 2037/0023; A61M 2037/0046; A61M 2037/0053

(57) ABSTRACT

A method of fabricating a microneedle is disclosed, including: applying a force to a conductive wire to create a friction weld between the wire and a substrate; extruding the wire and interrupting the wire bonding process; and applying a wire weakening process at a desired microneedle length to cause the wire to break at the desired microneedle length. A microneedle array includes a substrate and a plurality of solid microneedles provided on the substrate. Adjacent microneedles may have different heights and different diameters. The substrate defines a plurality of microfluidic channels each having a channel outlet. the channel outlets provided adjacent the bases of the plurality of solid microneedles to enable drug delivery. The method and array overcome issues with needle stick injuries and needle phobia, and can be used without direct medical supervision, thus reducing healthcare expenditure.

### FIELD

The present disclosure relates to hypodermic needles, including but not limited to microneedle arrays and systems and methods for fabricating a microneedle array.

### BACKGROUND

Transdermal drug delivery accounts for approximately 12% of all drug administrations, with the most prevalent method being the use of hypodermic needles. One in 10 Americans suffer from needle phobia, leading to reluctance or outright refusal in seeking medical care, worsening patient health outcomes and increasing healthcare spending. Rates of needle phobia increase significantly in youth. Recurring needle procedures throughout childhood can lead to needle phobia, in which higher rates of fear surrounding needles prompt higher perceived pain rates. Interventions in this cycle will overall increase quality of life and quality of healthcare for patients.

Additionally, needle stick injuries suffered by health care practitioners account for approximately 80% of all percutaneous injuries (injuries which break the skin barrier) and are extremely common in people managing chronic diseases with self-injections. These injuries are painful and leave patients and caregivers open to transmission of infectious diseases.

Microneedles are designed to offer a cost effective, low trauma, and high patient comfort alternative to injections administered using traditional hypodermic needles. However, microneedles have significant concerns with respect to scaling and manufacturing.

Improvements in approaches for fabricating microneedle arrays are desirable.

### SUMMARY

One aspect of the present disclosure relates to a method of fabricating a microneedle, including: applying a force to a conductive wire to create a friction weld between the wire and a substrate; extruding the wire and interrupting the wire bonding process; and applying a wire weakening process at a desired microneedle length to cause the wire to break at the desired microneedle length. The method may include breaking the wire at the desired microneedle length, for example using a micromanipulator, to create the microneedle. Applying the wire weakening process may include applying a weakening force to cause stress in the wire at the desired microneedle length. Applying the wire weakening process may include applying the wire weakening process using a micromanipulator. Applying the wire weakening process may include rapidly moving a bonder head of the wire bonder to a return position. Applying the wire weakening process may include applying a pulling force using an automated or semi-automated programmable wire bonder. The conductive wire may include a metal wire, such as a gold wire.

Another aspect of the present disclosure relates to a microneedle array, comprising a substrate defining a base layer material suitable for a wire bonding process, and a plurality of solid microneedles provided on the substrate. The plurality of solid microneedles may be fabricated in accordance with the methods as described and illustrated herein. The substrate may defines a plurality of microfluidic channels each having a channel outlet, the channel outlets provided adjacent the bases of the plurality of solid microneedles to enable drug delivery.

The method and array overcome issues with needle stick injuries and needle phobia, and can be used without direct medical supervision, thus reducing healthcare expenditure. The microneedle array may further include a control channel configured to enable selective activation of a subset of the plurality of solid microneedles. The control channel may be configured to send individually addressable signals to the plurality of solid microneedles to enable electroporation of epidermal cells of a skin surface to increase uptake of the drug, or may be used for iontophoresis to increase drug perfusion rates.

A controller may be provided in communication with the microneedle array and configured to provide a control signal on the control channel for selective activation of the subset of the plurality of solid microneedles. In an example implementation, the array comprises more than one microfluidic hole and channel in the substrate, for example adjacent or next to the microneedle or surrounding the microfluidic hole, enabling more than one drug type to be perfused in the space near to the microneedle.

A further aspect of the present disclosure relates to a method of fabricating a microneedle array, comprising, for a plurality of microneedles in the microneedle array: applying a force to a conductive wire to create a friction weld between the wire and a substrate; extruding the wire and interrupting the wire bonding process; and applying a wire weakening process at a desired microneedle length to cause the wire to break at the desired microneedle length.

Applying the wire weakening process may comprise, for a set of adjacent microneedles in the plurality of microneedles: controlling a path of a wire bonding head to create first and second microneedles in the set of adjacent microneedles such that a first microneedle has a first height and the second microneedle has a second height.

The method of fabricating a microneedle array may further comprise: obtaining a first conductive wire having a first diameter to form a first diameter microneedle; and obtaining a second conductive wire having a second diameter different from the first diameter to form a second diameter microneedle.

### BRIEF DESCRIPTION OF THE DRAWINGS

Embodiments of the present disclosure will now be described, by way of example only, with reference to the attached Figures.

FIG. 1 is a flowchart illustrating a method for fabricating a microneedle in accordance with one or more embodiments.

FIG. 2 illustrates outputs after completing different steps in a method of fabricating a microneedle, or a microneedle array, in accordance with one or more embodiments.

FIG. 3 illustrates a tip of a microneedle fabricated in accordance with one or more embodiments.

FIG. 4 illustrates a solid microneedle apparatus, in accordance with one or more embodiments.

FIG. 5 is a block diagram illustrating a microneedle array in accordance with one or more embodiments.

FIG. 6 illustrates a flexible substrate on which an array of solid individually electrically addressable microneedles may be fabricated, in accordance with one or more embodiments.

FIG. 7 illustrates an enlarged view of a plurality of microneedles fabricated on a rigid substrate, in accordance with one or more embodiments.

FIG. 8 illustrates an array of solid microneedles fabricated on a rigid substrate, in accordance with one or more embodiments.

FIG. 9 illustrates a flexible substrate and a microfluidic chip which cooperate to provide a microneedle and microfluidic system, in accordance with one or more embodiments.

FIG. 10 illustrates a microneedle and microfluidic system comprising the flexible substrate and the microfluidic chip of FIG. 9, in accordance with one or more embodiments.

FIG. 11 is an enlarged view of a portion of FIG. 10 showing the array of individually addressable microneedles and the related electrical connections.

FIG. 12 illustrates a glass or solid substrate comprising a plurality of microneedles and a microfluidic hole, in accordance with one or more embodiments.

FIG. 13 illustrates the substrate of FIG. 12 inserted into an adapter for a larger system, in accordance with one or more embodiments.

FIG. 14 shows the elements of FIG. 13 integrated with one another, in accordance with one or more embodiments.

FIG. 15 illustrates a syringe including the elements of FIG. 14, in accordance with one or more embodiments.

### DETAILED DESCRIPTION

A method of fabricating a microneedle is disclosed, including: applying a force to a conductive wire to create a friction weld between the wire and a substrate; extruding the wire and interrupting the wire bonding process; and applying a wire weakening process at a desired microneedle length to cause the wire to break at the desired microneedle length. A microneedle array includes a substrate and a plurality of solid microneedles provided on the substrate. Adjacent microneedles may have different heights and different diameters. The substrate defines a plurality of microfluidic channels each having a channel outlet, the channel outlets provided adjacent the bases of the plurality of solid microneedles to enable drug delivery. The method and array overcome issues with needle stick injuries and needle phobia, and can be used without direct medical supervision, thus reducing healthcare expenditure.

Known approaches using hypodermic needles have many drawbacks, such as insertion pain, tissue trauma, and expertise needed to perform an injection. Microneedle arrays provide advantages such as painless extraction and infusion by penetrating only the upper part of the skin, avoiding the nerves.

Solid microneedles, the most common type after hollow microneedles, are typically coated with a therapeutic agent, allowing the drug molecules to dissolve into the surrounding tissue after penetration of the skin barrier. The dosage depends on the microneedle area and therefore the yield is limited.

Solid microneedles are more physically robust than the more traditional hollow ones. However, their method of delivery, via drugs coated onto the microneedle's surface dissolving into the surrounding tissue, minimizes their effective dosage. Known microneedles have significant scaling, manufacturing, and drug efficacy concerns. Even though there are systems currently being developed and designed to deliver vaccinations for diseases such as hepatitis B, COVID-19, and Measles-Rubella, these have significant hurdles in developing processes to effectively pre-load systems with vaccinations that are typically reconstituted shortly before injection. In the cases where freeze drying is possible, larger scale up of polymer microneedles are a concern as the drug coating process is complex and inaccurate.

There is a need for sustainable, cost-effective, mass manufacturing of microneedles before they will see widespread adoption in medical systems.

Embodiments of the present disclosure provide a method for fabricating a solid microneedle array with embedded microfluidic channels in the substrate located next to the base of each microneedle that will allow delivery of relevant amounts of drugs through a pumping mechanism that is not limited to the area of the coated microneedles. A channel is described here as any perforation of an otherwise solid material or tube through which fluids may travel.

Microfabrication methods are well suited to create solid microneedle arrays, as the materials are biocompatible, robust and designed for large-scale integration with other micro manufacturing processes.

Embodiments of the present disclosure provide a method to fabricate solid microneedle arrays, for example using a manual wire bonder, a semi-automated wire bonder, or an automated wire bonder. An embodiment using an automated wire bonder allows for efficient and reliable fabrication of new designs of large area, high-density arrays in a repeatable, reliable and timely process.

Embodiments of the present disclosure provide a solid microneedle system that can be used without direct medical supervision, thus reducing healthcare expenditure.

For the purpose of promoting an understanding of the principles of the disclosure, reference will now be made to the features illustrated in the drawings and specific language will be used to describe the same. It will nevertheless be understood that no limitation of the scope of the disclosure is thereby intended. Any alterations and further modifications, and any further applications of the principles of the disclosure as described herein are contemplated as would normally occur to one skilled in the art to which the disclosure relates. It will be apparent to those skilled in the relevant art that some features that are not relevant to the present disclosure may not be shown in the drawings for the sake of clarity.

Certain terms used in this application and their meaning as used in this context are set forth in the description below. To the extent a term used herein is not defined, it should be given the broadest definition persons in the pertinent art have given that term as reflected in at least one printed publication or issued patent. Further, the present processes are not limited by the usage of the terms shown below, as all equivalents, synonyms, new developments and terms or processes that serve the same or a similar purpose are considered to be within the scope of the present disclosure.

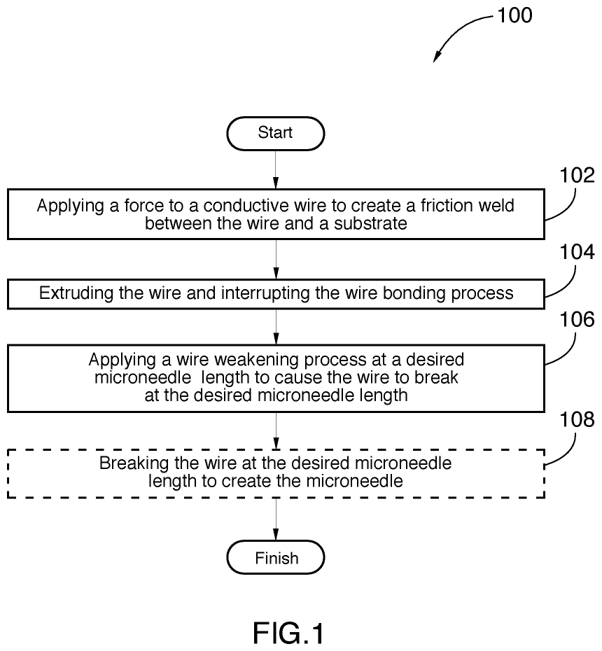

FIG. 1 is a flowchart illustrating a method 100 for fabricating a microneedle in accordance with one or more embodiments. The operations of method 100 presented below are intended to be illustrative. In some embodiments, method 100 may be accomplished with one or more additional operations not described, and/or without one or more of the operations discussed. Additionally, the order in which the operations of method 100 are illustrated in FIG. 1 and described below is not intended to be limiting.

In some embodiments, method 100 may be implemented in conjunction with one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 100 in response to instructions stored electronically on an electronic storage medium. The one or more processing devices may include one or more devices configured through hardware, firmware, and/or software to be specifically designed for execution of one or more of the operations of method 100.

An operation 102 may include applying a force to a conductive wire to create a friction weld between the wire and a substrate. Operation 102 may be performed by one or more hardware processors configured by machine-readable instructions to operate an apparatus such as a wire bonder, in accordance with one or more embodiments.

Applying the force may comprise applying a thermosonic process or applying an ultrasonic vibration. Creating the friction weld may comprise electroforming, using a wire bonder, the wire into a ball shape and pressing the wire into the substrate; this may be referred to as ball bonding. Creating the friction weld may alternatively comprise wedge bonding, which does not require or include ball formation. The wire may be a conductive wire, such as a metal wire, for example a gold wire.

An operation 104 may include extruding the wire and interrupting the wire bonding process. Operation 104 may be performed by one or more hardware processors configured by machine-readable instructions to operate an apparatus, such as a wire bonder, configured to extrude the wire, in accordance with one or more embodiments.

An operation 106 may include applying a wire weakening process at a desired microneedle length to cause the wire to break at the desired microneedle length. Operation 106 may be performed by one or more hardware processors configured by machine-readable instructions to operate an apparatus, such as a wire bonder, configured to apply the wire weakening process, in accordance with one or more embodiments. Applying the wire weakening process may comprise applying a weakening force to cause stress in the wire at the desired microneedle length. Applying the wire weakening process may comprise rapidly moving a bonder head of the wire bonder to a return position. Applying the wire weakening process may comprise applying a pulling force using an automated programmable wire bonder.

An operation 108 may include breaking the wire at the desired microneedle length to create the microneedle. Breaking the wire may comprise applying the wire weakening process using a micromanipulator. Operation 108 may be performed by one or more hardware processors configured by machine-readable instructions to operate an apparatus, such as a wire bonder, configured to extrude the wire, and further configured to break the wire at the point of wire weakening, in accordance with one or more embodiments.

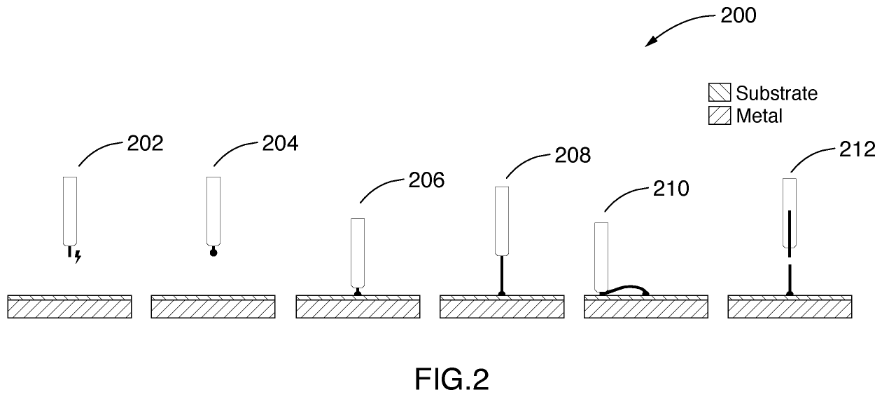

Embodiments of the present disclosure, for example as described in relation to FIG. 1, take the traditional wire bonding process and interrupt the process part way through to form vertical, solid microneedles, as will be described later in relation to FIG. 2. In an example embodiment, the wire from the bonder is attached to the base as normal, and then extruded upwards to a desired height. Instead of moving the wire to the next location to complete the traditional bond, a wire weakening process is applied at a desired microneedle length, causing the wire to break at the desired microneedle length and create the microneedle. In an example embodiment, using an automated wire bonder can make single microneedles in about 1 second, and enables building repeatable systems that are important for accuracy in testing and analysis.

In another embodiment, the present disclosure provides a method of fabricating a microneedle array comprising, for a plurality of microneedles in the microneedle array, operations as shown and described above in relation to FIG. 1, including: applying a force to a wire to create a friction weld between the wire and a substrate; extruding the wire and interrupting the wire bonding process; and applying a wire weakening process at a desired microneedle length to cause the wire to break at the desired microneedle length.

In an example embodiment, the method may fabricate a microneedle array in which adjacent microneedles have a different height. Known approaches are unable to perform such fabrication, since the microneedles are produced in batches, and the fabrications are not as flexible as those described herein. According to one or more embodiments, applying the wire weakening process comprises, for a set of adjacent microneedles in the plurality of microneedles: controlling a path of a wire bonding head to create first and second microneedles in the set of adjacent microneedles such that a first microneedle has a first height and the second microneedle has a second height. In this way, the microneedle array may be fabricated including adjacent microneedles of different heights, which may be advantageous for different implementations and approaches of drug delivery.

In an example embodiment, the method may fabricate a microneedle array in which adjacent microneedles have a different diameter. In such an embodiment, the method comprises: obtaining a first conductive wire having a first diameter to form a first diameter microneedle; and obtaining a second conductive wire having a second diameter different from the first diameter to form a second diameter microneedle. Using a semi-automated or automated wire bonder, embodiments of the present disclosure can select wires having different diameters to fabricate a microneedle array including adjacent microneedles of different diameter or thickness, which may be advantageous for different implementations and approaches of drug delivery.

FIG. 2 illustrates outputs after completing different steps in a method of fabricating a microneedle, or a microneedle array, in accordance with one or more embodiments. At 202, a step of electroforming is shown, for example using a wire bonding capillary. In an example implementation, at 202 a small amount of wire may be extruded and a few kilovolts applied to the wire to melt the wire. At 204, a metal ball is shown has having been formed, for example using the electroforming process. At 206, a capillary head comes down and contacts the surface of the metal on the substrate, performing a bond, such as a thermosonic bond. At 208, the wire is being extruded, for example by retracting the capillary without holding on to the wire.

At 210, the wire is clamped again and moved to the side to impinge the wire against the metal surface. In an implementation, the capillary or bond head is moved down and then across, but in another implementation the bond head may be moved back slightly or another movement performed to cause the weakening. The path of the capillary may be varied based on the material used and the desired physical properties of the microneedle. In contrast to known approaches, the process is such that another thermosonic bond is not created, but rather the process begins to create a weak point in the wire by compressing the wire at the point where another thermosonic bond would typically be created using wire bonding. In an implementation, enough pressure is applied to make the wire weak, but not enough pressure to bond the wire to the metal. At 212, the capillary is retracted to the original position, such that the weak point where the compression was applied separates and creates a gap, thus forming the tip of a microneedle.

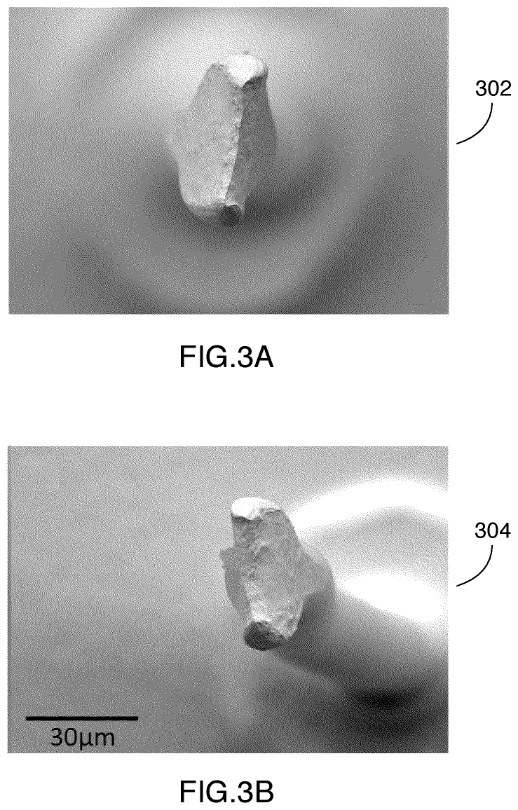

FIG. 3 illustrates a tip of a microneedle fabricated in accordance with one or more embodiments. FIG. 3 provides scanning electron microscope images showing 25 μm gold wirebonded microneedles. In an example implementation, microneedles were inserted into a dense foam designed to mimic the bulk tissue properties of the dermis. At 302, the microneedle is illustrated before insertion into the dense foam. At 304, the microneedle is illustrated after insertion. In the example embodiment of FIG. 3, the shaft of the microneedle is about 500 μm tall.

A method according to an embodiment of the present disclosure fabricates a solid microneedle array using automatable, scalable processes capable of delivering pharmaceuticals across the dermis as an effective replacement for several forms of hypodermic injection. Common existing practices for creating commercially viable microneedle arrays include: laser micromachining, a process which is slow and requires further processing to convert a 2d structure into a 3d structure; thermoforming/micro-molding, which requires the use of complex photolithographic techniques to create base molds; and micro stereolithography/2photon-polymerization, which is time consuming. None of these technologies meet the criteria that would bring microneedles to the forefront of healthcare, which is an adaptable, high speed, low cost method.

Embodiments of the present disclosure use an ultrasonic or thermosonic wirebonder (for example, 56 Series, F&S Bondtec) to provide a method by which solid microneedles can be rapidly created. In an example embodiment, a gold wire, 25 micrometers thick, may be electroformed into a ball shape, which is then pressed into the substrate. When an ultrasonic vibration is applied, a friction weld is created. The wire is then extruded and a process to weaken the wire is applied at the desired microneedle length. In an example implementation, the bonder head is rapidly moved to a return position, snapping the wire at the weak point, leaving the crescent shape tip as seen in FIG. 3. These bonds may be automated and formed extremely rapidly, producing a microneedle array at speeds of about 1 needle per second. This method rivals even most common high-speed manufacturing methods such as thermoforming and laser micromachining. Importantly, this method requires minimal user intervention or involvement, and does not require dedicated infrastructure such as cleanrooms or photolithography suites. A wirebonding system may be customized to create variable configurations, densities, and heights with minimal changes to processes.

A metal microneedle according to an embodiment of the present disclosure, as shown in FIG. 3, is robust and has minimal wear and damage to the microneedle post-injection. Existing polymer and silicon based microneedles face challenges in fracture, shearing, and deposition of needle material in the dermis, leading to granulomas and localized irritation. Embodiments of the present disclosure may include a metal microneedle, for example formed of gold wire. The material properties of gold, notably the ductility, means that there is little risk of leaving debris embedded in the skin. Wirebonding is a technique developed to work with integrated circuits, which enables integration of electrical and microneedle systems. By electrically connecting all wires, a microneedle array according to embodiments of the present disclosure may be integrated with iontophoretic or electrophoretic systems to increase drug transfusion into the skin or increase cellular uptake of pharmaceuticals. Additionally, in an example embodiment, the use of gold wires provides capacity for simple functionalization and adaptation to lab-on-a-chip technologies that rely on antibody or aptamer detection of in vivo substances.

Table 1 and Table 2 below provide example parameters for a method of fabricating a microneedle, in accordance with one or more embodiments. The parameters are from methods performed using 25 micron gold wire on a FR24 PCB (printed circuit board) substrate with an ENEPIG (Electroless Nickel Electroless Palladium Immersion Gold) coating, and completed on a TPT HB16 semi-automatic wire bonder, at a heater temperature of 95 degrees Celsius. The method in this example, and using the loop parameters in Table 2 for the forming and extrusion, fabricated microneedles with a length of approximately 415 μm.

TABLE 1

| | Bond 1 | Bond 2 |
|---|---|---|
| US (arbitrary unit) | 220 | 200 |
| Time (ms) | 200 | 50 |
| Force (formulaic value) | 200 | 250 |

TABLE 2

| Loop parameters (μm) | |
|---|---|
| Up | 250 |
| Back | 150 |
| Up | 150 |
| Front | 450 |
| Down | 300 |

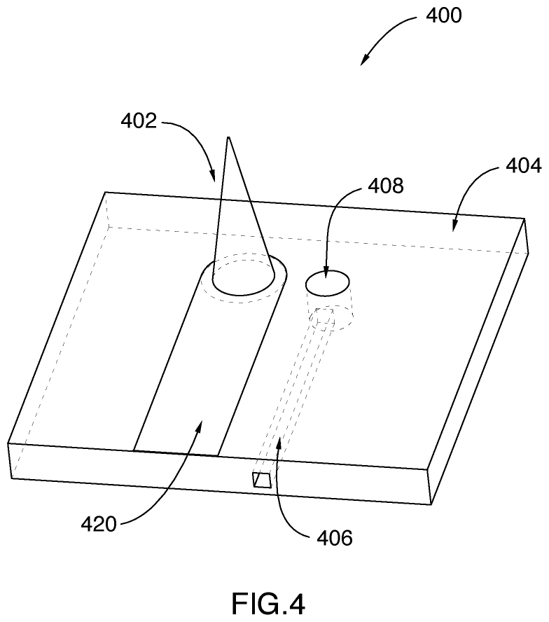

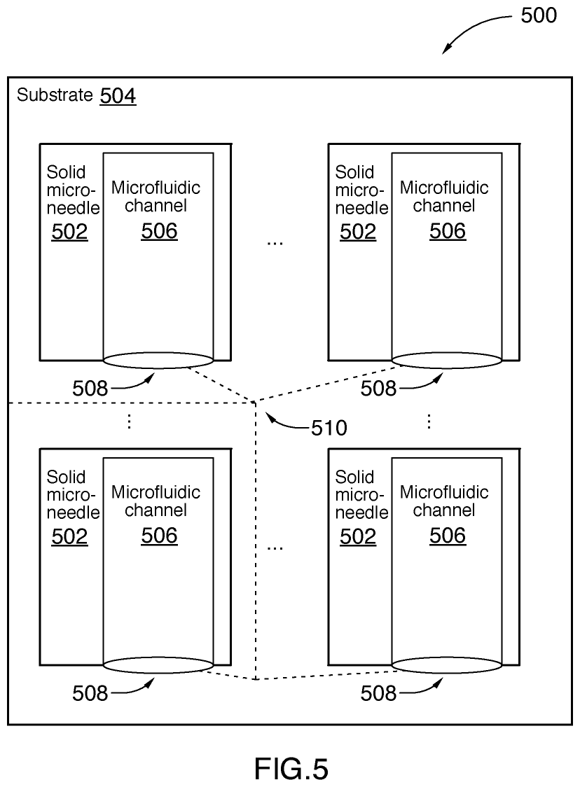

FIG. 4 and FIG. 5 illustrate examples of solid microneedles and solid microneedle arrays with individual electrical connections, with an adjacent microfluidic channel for fluid delivery, in accordance with one or more embodiments.

FIG. 4 illustrates a solid microneedle apparatus or system 400, in accordance with one or more embodiments. The system 400 comprises a solid microneedle 402 and a substrate 404. Each solid microneedle 402 has a tip and a base, and is provided on the substrate. The tip and the base may be at opposing ends of the solid microneedle 402. The substrate 404 defines a microfluidic channel 406, including an interface or outlet 408. As shown in FIG. 4, a microfluidic channel outlet 408 may be provided adjacent the base of the solid microneedle 402.

In the example embodiment of FIG. 4, the system further comprises a control channel or electrical connection 420 configured to enable selective activation of the solid microneedle, for example by carrying or transmitting an electrical signal. In an example embodiment, the control channel is configured to send an individually addressable signal to the solid microneedle to enable electroporation of epidermal cells of a skin surface to increase uptake of the drug.

FIG. 5 illustrates a solid microneedle system 500, for example as may be provided in a microneedle array in accordance with one or more embodiments. The system 500 comprises a plurality of solid microneedles 502 and a substrate 504. Each solid microneedle 502 has a tip and a base, and is provided on the substrate. The tip and the base may be at opposing ends of the solid microneedle 502. The substrate 504 defines a plurality of microfluidic channels 506, each including an interface or outlet 508. As shown in FIG. 5, a microfluidic channel outlet 508 may be provided adjacent the base of the solid microneedle 502, and each solid microneedle may have an adjacent microfluidic channel outlet 508.

In the embodiment of FIG. 5, the substrate 504 defines a plurality of apertures 508 adjacent the bases of the plurality of solid microneedles 502, and in communication with the plurality of microfluidic channels 506 to enable drug delivery. The plurality of microfluidic channels 506 may be in communication with each other via a microfluidic system 510, which may be a shared microfluidic connection. The microfluidic system 510 may be considered to provide one inlet to the plurality of microfluidic channel outlets 508. The example of FIG. 5 includes an array of individual solid microneedles with a microfluidic channel and an outlet directly next to the microneedle.

In an example embodiment (not shown), the microneedle array 500 of FIG. 5 may further comprise a control channel or electrical connection, similar to the control channel 420 of FIG. 4, configured to enable selective activation of a subset of the plurality of solid microneedles, for example by carrying or transmitting an electrical signal. A control channel implemented in the embodiment of FIG. 5 may include a plurality of electrical connections in a network, or any other combination of control connections able to carry a control signal, such as an electrical control signal. In an example embodiment, the control channel is configured to send individually addressable signals to the plurality of solid microneedles to enable electroporation of epidermal cells of a skin surface to increase uptake of the drug. In an example embodiment, a controller is in communication with one or more microneedles 502 in a microneedle array and configured to provide a control signal on the control channel for selective activation of the subset of the plurality of solid microneedles.

In an example implementation, the array comprises more than one microfluidic hole and channel in the substrate, for example adjacent or next to the microneedle or surrounding the microfluidic hole, enabling more than one drug type to be perfused in the space near to the microneedle. In an example embodiment, the microfluidic channel outlet is adjacent the microneedle and configured such that the microfluidic channel may run parallel to the electrical connection or may be connected to a larger reservoir elsewhere.

In an embodiment, the present disclosure provides a microneedle array, comprising: a substrate defining a base layer material suitable for a wire bonding process; and a plurality of solid microneedles provided on the substrate and fabricated in accordance with any one of the methods of fabricating a microneedle as described and illustrated herein. The substrate may be either rigid or flexible. The substrate may comprise a plurality of microfluidic channels each having a channel outlet, the channel outlets provided adjacent bases of the plurality of solid microneedles to enable drug delivery.

In an example embodiment, the microneedle array further comprises a control channel configured to enable selective activation of a subset of the plurality of solid microneedles. In an example embodiment, the control channel is configured to send individually addressable signals to the plurality of solid microneedles to enable electroporation of epidermal cells of a skin surface to increase uptake of the drug. In an example embodiment, the control channel is configured to send individually addressable signals to the plurality of solid microneedles to enable iontophoresis of pharmacological compounds to increase uptake of the drug. In an example embodiment, the microneedle array further comprises a controller configured to provide a control signal on the control channel for selective activation of the subset of the plurality of solid microneedles.

In an example embodiment, the control channel is configured to receive signals from the plurality of solid microneedles to enable detection of biosignals or electrical activity due to biological interactions with a coating on the plurality of solid microneedles. In an example embodiment, one or more microneedles in the microneedle array are coated in a bioresorbable compound capable of drug delivery.

The microneedle array may be is affixed to an external system, such as a syringe, that facilitates one or more of: drug delivery; processing of biosignals; and microfluids testing systems.

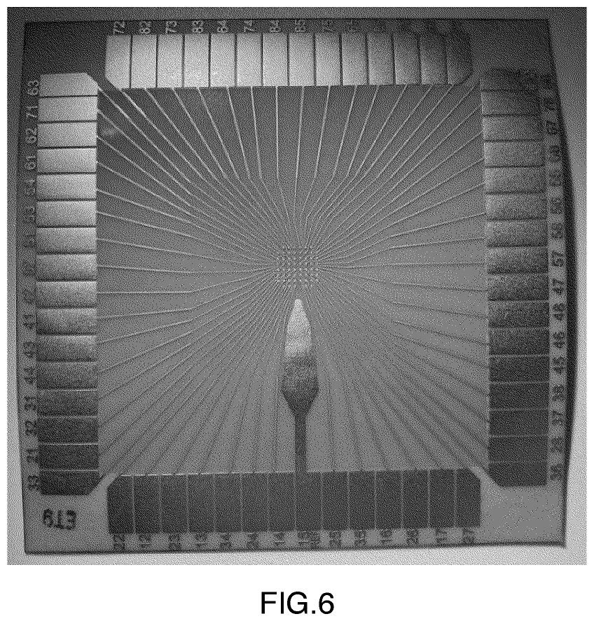

FIG. 6 illustrates a flexible substrate on which an array of solid individually electrically addressable microneedles may be fabricated, in accordance with one or more embodiments. Embodiments of the present disclosure provide an improvement over existing processes, by providing a fabrication method using low cost commercial flexible printed circuit boards (PCBs), for example as shown in FIG. 6. Based on experimental and theoretical simulation of the microneedle physical structures, embodiments of the present disclosure may consider material and design considerations for mechanical stability, biocompatibility, and reproducibility.

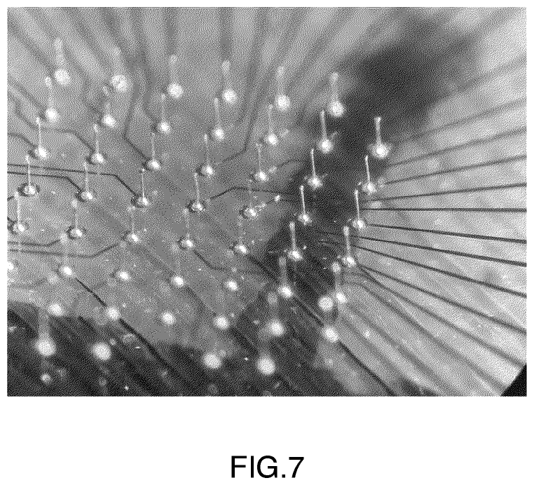

FIG. 7 illustrates an enlarged view of a plurality of microneedles fabricated on a rigid substrate, in accordance with one or more embodiments. The example embodiment of FIG. 7 shows a plurality of microneedles formed on a glass substrate.

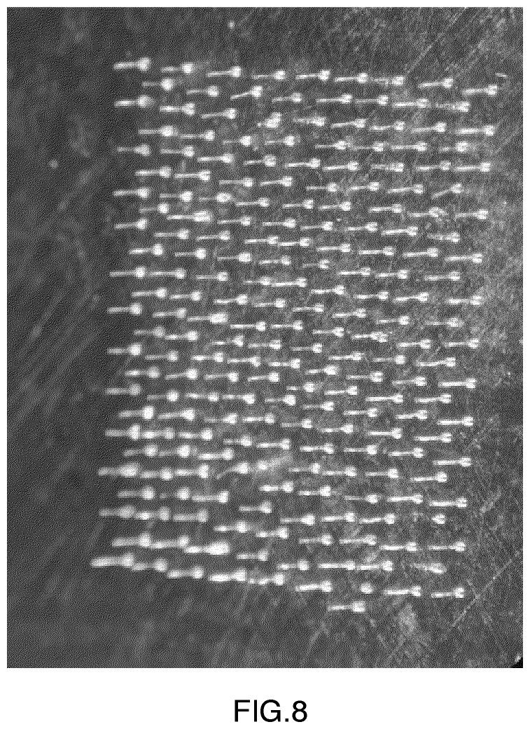

FIG. 8 illustrates an array of solid microneedles fabricated on a rigid substrate, in accordance with one or more embodiments. In an example embodiment, as shown in FIG. 8, the rigid substrate provides a single electrically conductive surface, such as a metal surface. In such an embodiment, the array does not need individually addressable electrical lines, and the microneedles may all be connected on one conductive surface. In another example embodiment, the rigid substrate may comprise a glass substrate, and a plurality of individual wires may be provided on the glass substrate to provide connectivity with the microneedles. While some embodiments described earlier included individually addressable microneedles, in the embodiment of FIG. 8 an array of microneedles may all be connected to a single electrical input, for example via the electrically conductive surface, and configured to detect an electrical impulse or another electrical control signal.

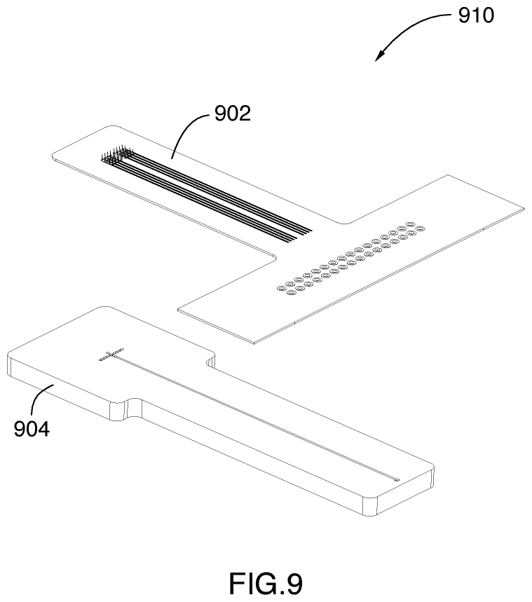

FIGS. 9-11 illustrate embodiments using a flexible substrate and a microfluidic chip.

FIG. 9 illustrates a flexible substrate 902 and a microfluidic chip 904 which cooperate to provide a microneedle and microfluidic system 910, in accordance with one or more embodiments. The flexible substrate 902 comprises an array of individually addressable solid microneedles, and is configured to be mated with, or applied to, a microfluidic chip 904. In an example implementation, the flexible substrate including the array of microneedles may be produced in accordance with a method of one or more embodiments described herein. The microfluidic chip 904 may be a commercially available item, with which the flexible substrate is designed to cooperate. Alternatively, the microfluidic chip 904 may be customized based on properties of the flexible substrate 902.

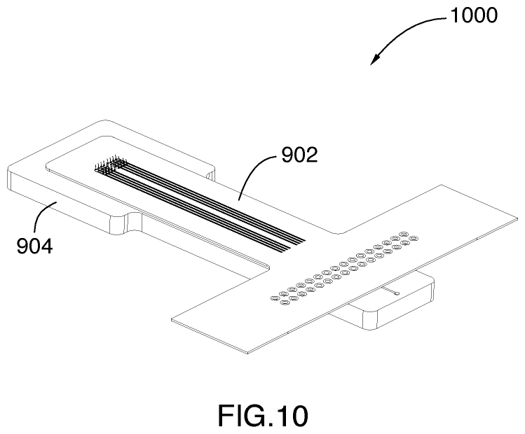

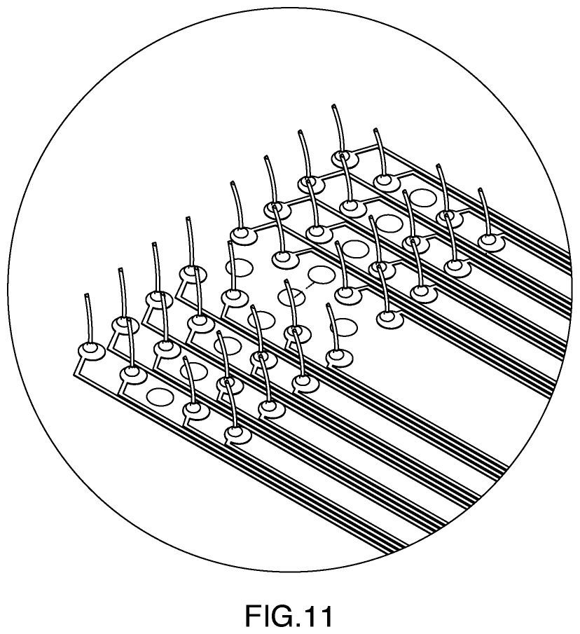

FIG. 10 illustrates a microneedle and microfluidic system 1000 comprising the flexible substrate 902 and the microfluidic chip 904 of FIG. 9, in accordance with one or more embodiments. In an embodiment, the flexible substrate is bonded or adhered to the microfluidic chip to produce an integrated device. In an embodiment, the array of microneedles is built on the flexible substrate, which is then deposited onto a larger device, such as the microfluidic chip. FIG. 11 is an enlarged view of a portion of FIG. 10 showing the array of individually addressable microneedles and the related electrical connections.

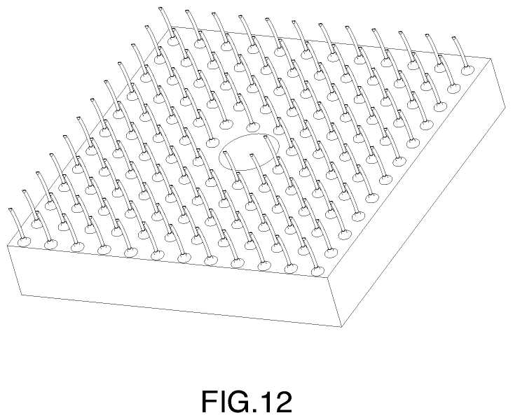

FIGS. 12-15 illustrate a solid microneedle array with a central fluidic channel to be affixed, by use of an adaptive system, to the head of a syringe for drug delivery, in accordance with one or more of the embodiments. FIG. 12 illustrates a glass or solid substrate comprising a plurality of microneedles and a microfluidic hole, in accordance with one or more embodiments. FIG. 12 is an example of, and may be described as, an insert defining a central aperture or hole surrounded by solid microneedles. The central aperture may enable fluid to be pumped into an area of perforated skin, and may be provided in an embodiment as a fluid outlet surrounded by concentric rings of microneedles.

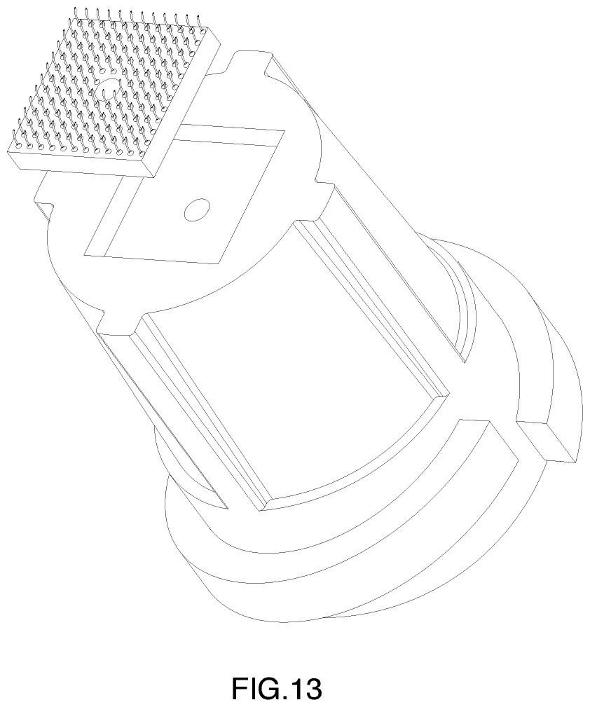

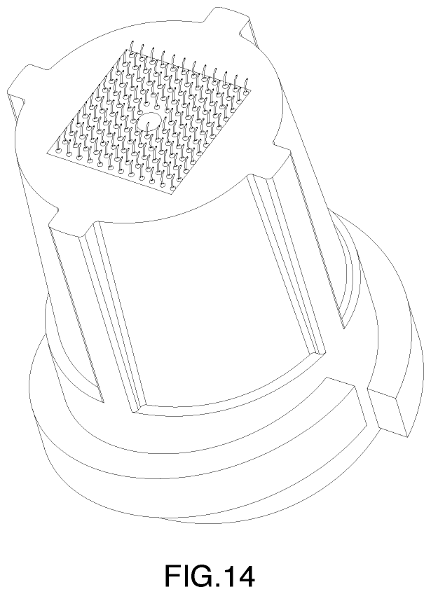

FIG. 13 illustrates the substrate of FIG. 12 configured for insertion into an adapter for a larger system, such as a Luer Lock microneedle syringe, in accordance with one or more embodiments. The substrate of FIG. 12 may be inserted into a 3D printed Luer-lock, which can replace the removable single macro needle on a typical syringe. The illustration in FIG. 13 shows alignment of the microfluidic hole with a hole or aperture in the adapter. FIG. 14 shows the elements of FIG. 13 integrated with one another, in accordance with one or more embodiments.

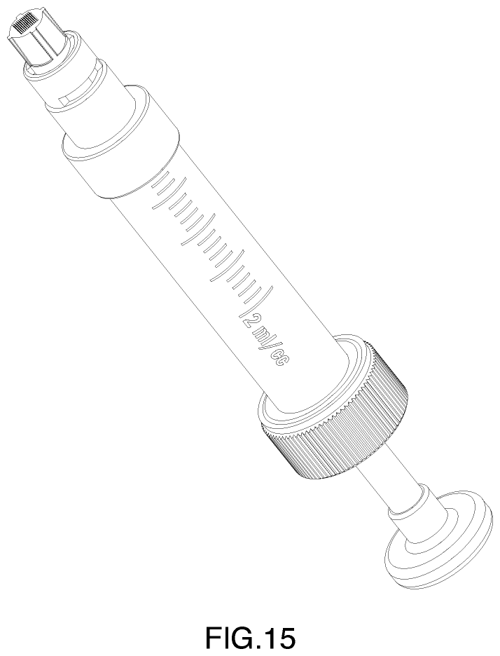

FIG. 15 illustrates a syringe including the elements of FIG. 14, in accordance with one or more embodiments. While the embodiments in FIGS. 12-15 do not include electrical connections, according to other embodiments electrical connections may be provided in the solid microneedle array, and enabled by one or more sources of electrical power and control, for example in association with the syringe or other delivery apparatus. The electrical connections may enable solid microneedles to be individually addressable, or addressable in groups.

In an example implementation, in order to quantify microneedle efficacy, arrays of varying density were inserted into tissue analogs using a materials testing machine (for example, H1KT, Tinius Olsen) with a 100 N load cell. Microneedle arrays were linearly driven towards the tissue analog at a rate of 100 μm/s until the needles had fully pierced the tissue. Force and position data were captured, which can be used to determine insertion forces during various stages of injection, determining when the skin is being tensioned, pierced, and when the needle array is fully inserted. It has been shown that insertion force is directly correlated to patient experienced pain during injection. Arrays of densities ranging between about 100 needles/cm² and about 1056 needles/cm² were analyzed for insertion forces. Tissue analogs were used to simulate insertion into human skin. Tissue substitutes used for analysis were a synthetic foam designed to replicate bulk dermis mechanical properties (3B Scientific) coated in a PDMS (Sylgard 184 Elastomer) layer designed to mimic the elastic and mechanical properties of the epidermal layer. The PDMS layer was deposited through the use of a spin coater to form long polymer chains in a thin (˜20 um) layer on the surface of the foam. Experimental results showed that these microneedle arrays have peak insertion forces on the range of about 1.0 to about 1.5 N when injecting into synthetic materials. These forces are comparatively low compared to forces experienced by similar size and density polymer needles, which reached forces of about 10 N to about 15N in the synthetic analog.

Using an automated wire bonder, embodiments of the present disclosure may provide rapid iterative development of solid microneedles, and may produce solid microneedles of different diameters and heights, in multiple materials, on multiple substrate types (both solid and flexible). Embodiments of the present disclosure avoid the need to create microneedles using traditional, time consuming and process-intensive methods. In a method according to an example embodiment, a wire bonder may be used to enable microneedles, and more importantly, microneedle arrays, to be fabricated, produced and developed in a timely and consistent manner.

In an example embodiment, an automated wire bonder comprises a wedge, ball and ribbon bonding capability, tail length control, heated workspace and programmable loop control. In an example embodiment, a solid microneedle may have a sharp, sub μm tip.

In another implementation, the microneedles may be about 17 μm in diameter and may range from 100 to 600 μm high, with 500 μm spacing shown. In an embodiment, a microneedle array comprises microfluidic channels leading to holes adjacent to the bases of the microneedles, which will enable better drug delivery than existing single use solid microneedle coating methods.

According to an embodiment of the present disclosure, a fabrication process interrupts the typical wire bonding cycle. In an example embodiment using a manual bonder, the manual bonder operational set up may need to be modified each time it is used, returning the equipment back to its correct settings afterwards. The location of the microneedle may be determined by application requirements, for example a dense array patch or more spaced microneedles. In an example embodiment, an automated micromanipulator process is provided with an optical feedback system that may be linked to a manual wire bonder. Other embodiments of the present disclosure provide an automated process to create solid microneedles by bending the wire after the bond to the substrate to cause a stress fracture in the wire, such that the wire is caused to break, or may be broken, at a set point, removing the need for manual cutting.

Embodiments of the present disclosure may be provided for a plurality of uses and in a plurality of scenarios. For example, a solid microneedle array may be provided on a substrate with fluidic channels, such as illustrated in many of the figures. A single hole or aperture may be provided, such as in the embodiments shown in FIGS. 12-16. Channels may be provided adjacent to microneedles, such as in the embodiments shown in FIGS. 4 and 5. A solid microneedle array produced in accordance with one or more embodiments may be affixed to a larger fluidic system, such as shown in FIGS. 9-11 and in FIGS. 12-16. Such implementations may be designed for insertion into tissue for the purposes of drug delivery.

In another example, a solid microneedle array may be provided on a substrate without fluidic channels. In one such an implementation, the microneedles may be inserted into tissue for topical drug ointment for drug delivery. In another such implementation, a polymeric coating may be provided to the microneedles to control or modify drug delivery upon insertion into tissue. The polymeric coating may enhance the ability of the microneedle to detect certain molecules.

Embodiments of the present disclosure may be provided for a plurality of uses and in a plurality of scenarios, relating to drug delivery and biosensing. For example, a microneedle array in accordance with one or more embodiments may comprise an electrically connected microneedle array, which may comprise bare metal electrodes or coated metal electrodes. Bare metal electrodes may be used for iontophoretic modulated drug delivery, electroporation modulated drug delivery, or biosensing. Coated metal electrodes may be used for iontophoretic modulated drug delivery, electroporation modulated drug delivery, electrically activated drug delivery or augmented biosensing.

Embodiments of the present disclosure provide the ability to make solid microneedles out of a variety of biocompatible materials, such as gold, with very sharp tips with a range of diameters, heights and spacing. These microneedles, being made of metals, have an added advantage of allowing individually addressable electrical signals to be sent to the microneedles to enable electroporation of the skin's epidermal cells to increase their uptake of drug. Using this feature, embodiments of the present disclosure have the ability to only activate certain microelectrodes/needles in areas as needed. Having localized electrical signals can also be used to activate certain drug types or custom polymers. For practical application of a solid microneedle array in a patch like scenario, the microneedle array may be flexible, to conform to the skin.

Embodiments of the present disclosure provide a method of fabricating solid microneedles that enable low-cost manufacture. Embodiments of the present disclosure provide a microneedle array, with built-in fluidic and electronic control, for implementation in the healthcare system. According to an embodiment of the present disclosure, a solid microneedle array includes a flexible substrate with integrated microfluidic channels to overcome the dosage issue for single use drug delivery.

In an example implementation, a desktop unit is provided with a disposable solid microneedle array insert on a rigid substrate where the thumb is pressed onto the array for drug delivery. In another example implementation, a solid microneedle array is provided on a flexible substrate with integrated microfluidic channels and reservoirs to overcome dosage issues for single use drug delivery, for example a drug delivery band aid or patch. In another example implementation, a solid microneedle array is provided on a prebuilt Luer-lock interface which can be readily attached to existing hypodermic needle platforms.

Embodiments of the present disclosure provide systems that improve upon current drug delivery practices and overcome issues with needle stick injuries and needle phobia.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof. As another example, specific details are not provided with respect to microfabrication processes, such as wire bonding.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a compact disk read only memory (CD-ROM), digital versatile disk (DVD), Blu-ray Disc Read Only Memory (BD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be effected to the particular embodiments by those of skill in the art without departing from the scope, which is defined solely by the claims appended hereto.

What is claimed is:

1. A method of fabricating a microneedle, comprising:
applying a force to a conductive wire to create a friction weld between the wire and a substrate;
extruding the wire and interrupting the wire bonding process; and
applying a wire weakening process at a desired microneedle length to cause the wire to break at the desired microneedle length.

2. The method of claim 1, wherein applying the force comprises applying a thermosonic process or applying an ultrasonic vibration.

3. The method of claim 1, wherein creating the friction weld comprises:
electroforming, using a wire bonder, the wire into a ball shape and pressing the wire into the substrate.

4. The method of claim 3, wherein applying the wire weakening process comprises:
rapidly moving a bonder head of the wire bonder to a return position.

5. The method of claim 1, wherein applying the wire weakening process comprises:
applying a weakening force to cause stress in the wire at the desired microneedle length.

6. The method of claim 1, wherein applying the wire weakening process comprises:
applying a pulling force using an automated programmable wire bonder.

7. The method of claim 1, further comprising:
breaking the wire at the desired microneedle length to create the microneedle.

8. The method of claim 7, wherein breaking the wire at the desired microneedle length comprises:
applying the wire weakening process using a micromanipulator.

9. A microneedle array, comprising:
a substrate defining a base layer material suitable for a wire bonding process; and
a plurality of solid microneedles provided on the substrate and fabricated in accordance with the method of any one of claims 1-8.

10. The microneedle array of claim 9 wherein the substrate comprises a plurality of microfluidic channels each having a channel outlet, the channel outlets provided adjacent bases of the plurality of solid microneedles to enable drug delivery.

11. The microneedle array of claim 9 further comprising:
a control channel configured to enable selective activation of a subset of the plurality of solid microneedles.

12. The microneedle array of claim 11 wherein:
the control channel is configured to send individually addressable signals to the plurality of solid microneedles to enable electroporation of epidermal cells of a skin surface to increase uptake of a drug.

13. The microneedle array of claim 11 wherein:
the control channel is configured to send individually addressable signals to the plurality of solid microneedles to enable iontophoresis of pharmacological compounds to increase uptake of a drug.

14. The microneedle array of claim 11 further comprising:
a controller configured to provide a control signal on the control channel for selective activation of the subset of the plurality of solid microneedles.

15. The microneedle array of claim 11 wherein:
the control channel is configured to receive signals from the plurality of solid microneedles to enable detection of biosignals or electrical activity due to biological interactions with a coating on the plurality of solid microneedles.

16. The microneedle array of claim 11, wherein at least some of the plurality of solid microneedles are coated in a bioresorbable compound capable of drug delivery.

17. The microneedle array of claim 11, wherein the microneedle array is configured to be affixed to an external system to facilitate one or more of: drug delivery; processing of biosignals; and microfluids testing systems.

18. A method of fabricating a microneedle array, comprising:
for a plurality of microneedles in the microneedle array:
applying a force to a conductive wire to create a friction weld between the wire and a substrate;
extruding the wire and interrupting the wire bonding process; and
applying a wire weakening process at a desired microneedle length to cause the wire to break at the desired microneedle length.

19. The method of claim 18, wherein applying the wire weakening process comprises, for a set of adjacent microneedles in the plurality of microneedles:
controlling a path of a wire bonding head to create first and second microneedles in the set of adjacent microneedles such that a first microneedle has a first height and the second microneedle has a second height.

20. The method of claim 18, further comprising:
obtaining a first conductive wire having a first diameter to form a first diameter microneedle; and
obtaining a second conductive wire having a second diameter different from the first diameter to form a second diameter microneedle.
